(19) United States
(12) Patent Application Publication
(10) Pub. No.: US 2018/0003613 A1
(43) Pub. Date: Jan. 4, 2018

(54) Phenotypic High-Content Assay to Evaluate Drugs

(71) Applicant: Texas Tech University System, Lubbock, TX (US)

(72) Inventor: P. Michael Conn, Lubbock, TX (US)

(21) Appl. No.: 15/543,581

(22) PCT Filed: Jan. 15, 2016
(86) PCT No.: PCT/US2016/013610

§ 371 Date: Jul. 14, 2017

Related U.S. Application Data
(60) Provisional application No. 62/103,889, filed on Jan. 15, 2015.

(51) Int. Cl. G01N 15/14; G01N 33/50; G01N 15/10; G01N 21/00; C12Q 1/00

(52) U.S. Cl. CPC G01N 15/1475; G01N 33/5035; G01N 33/5038; C12Q 1/00; G01N 21/00; G01N 2015/1006

(57) ABSTRACT

The present invention includes a high throughput screen for an active agent for the treatment of comprising: plating cells at least one pathophysiologically relevant mislocated mutant form of a peroxisomal enzyme; adding a control and compound to each plate from a library of compounds; fixing the cells; contacting the cells with an agent that detects the mislocated mutant form of a peroxisomal enzyme; and imaging the cells in the wells.

### TECHNICAL FIELD OF THE INVENTION

The present invention relates in general to the field of phenotypic screening methods, and more particularly, to a method for screening drugs for phenotype by high-throughput screening.

### BACKGROUND OF THE INVENTION

Without limiting the scope of the invention, its background is described in connection with Oxalosis and Hyperoxaluria.

Primary hyperoxaluria is a severe disease for which the best current therapy is dialysis or organ transplantation. These are risky, inconvenient, and costly procedures. In some patients pyridoxine treatment can delay the need for these surgical procedures. The underlying cause of particular forms of this disease is the misrouting of a specific enzyme; alanine:glyoxylate aminotransferase (AGT) to the mitochondria instead of the peroxisomes.

Primary hyperoxaluria (PH) is a rare autosomal recessive hereditary disorder responsible for an excessive production of oxalate that progressively accumulates as calcium salts and forms renal and/or bladder stones. As it progresses, the disease can lead to chronic kidney failure and systemic oxalosis (deposition of calcium oxalate throughout the body). Deficiencies in the liver-specific peroxisomal enzyme alanine:glyoxylate aminotransferase (AGT, EC 2.6.1.44) has been linked to Type 1 PH. Under normal circumstances, AGT catalyzes the transamination of glyoxylate to glycine within the peroxisomes of hepatocytes. In PH1 patients, who represent 80% of the PH population, the AGT deficiency causes the glyoxylate to diffuse from the peroxisomes into the cytosol, where it is oxidized to insoluble calcium oxalate by the lactate dehydrogenase. Missense mutations in the AGT gene (AGXT) that lead to the mistargeting of AGT from its normal peroxisomal location to the mitochondria have been identified. Although mistargeted, AGT is still catalytically active in the mitochondria but metabolically ineffective in this location. The best therapies currently rely on dialysis or liver transplantation; both represent inconvenient, risky and costly procedures. Hence, cellular rescue of AGT misrouting via small molecule intervention is a promising and potentially safer therapeutic alternative.

One example of a pharmacoperone is taught in U.S. Pat. No. 7,842,470, filed by Conn entitled, “Method for pharmacoperones correction of GnRHR mutant protein misfolding.” Briefly, the invention is said to relate to methods of identifying pharmacoperone agents that can restore function to a misfolded protein, such as a misfolded protein that causes disease. The patent is also said to disclosed methods of using such pharmacoperone agents to treat a disease or disorder that results from the misfolded protein.

Another example is taught in United States Patent Application Publication No. 20100317690, filed by Kawamura, et al., entitled, “Treatment of Protein Folding Disorders.” Briefly, these applicants are said to teach various compounds and methods for the treatment of disorders arising from aberrant protein folding, in particular lysosomal storage diseases. Polyhydroxylated alkaloids and imino sugars are said to be pharmacoperones of an enzyme and that do not bind to a catalytic site of the enzyme.

### SUMMARY OF THE INVENTION

One embodiment of the present invention includes a method of determining the effectiveness of one or more drug candidates to change the intracellular localization of a target molecule, the method comprising: (a) incubating the one or more drug candidates with a first subset of the cells, and a control agent with a second subset of the cells; (b) fixing and staining the first and second subset of cells, wherein the stain detects the target molecule; (c) generating images of the first and second subset of cells with a camera; (d) measuring the difference in the intracellular localization of the target molecule in the first as compared to a second subset of cells; and (e) determining if the drug candidate modifies the localization of the intracellular localization of the target protein, wherein if the candidate drug modifies the intracellular localization of the target protein when compared to the placebo it is an effective drug candidate. In one aspect, the method is defined further as calculating a range of localization values are assigned a value ranging from −1 to 1, which is the degree of overlap of the two targets with each other independent of the intensity differences of the two targets, and is calculated using the following equation:

$${r}_{p} = \frac{\sum ( x - \dot{x} ) ( y - \dot{y} )}{\sqrt{\sum {( x - \dot{x} )}^{2} {( y - \dot{y} )}^{2}}}$$

Where rₚ is the Pearson's correlation coefficient, x and y are pixel intensities of each pixel detected for the target protein in the first versus the second subset of cells, respectively, and x and y are average pixel intensities of the puncta identified as a position of the target protein in the first versus the second subset of cells, respectively. In another aspect, the values are normalized on a per plate basis using the following equation:

$$\% rescue = 100 \times \frac{Test Well - Median Low Control}{Median High Control - Median Low Control} .$$

In another aspect, the method further comprises the step of determining cell count, nuclear intensity, morphology and condensation. In another aspect, the localization changes from the cytosol or mitochondria to a peroxisome. In another aspect, the candidate agent is selected from at least one of 26-Deoxymonensin B, nigericin, salinomycin, or active derivatives thereof. In one aspect, the condition is Oxalosis or Hyperoxaluria, such as Type 1 Hyperoxaluria. In one aspect, the candidate agent correctly folds and/or routes otherwise-misfolded/mistrafficked mutant proteins to the correct intracellular or extracellular location.

Another embodiment of the present invention includes a method of determining the effectiveness of one or more candidate pharmacoperones to treat and/or prevent protein misfolding, the method comprising: (a) incubating the one or more candidate pharmacoperones with a first subset of the cells, and a placebo with a second subset of the cells; (b) fixing and staining the first and second subset of cells, wherein the stain detects anti-AGT in the cells; (c) generating images of the first and second subset of cells with a camera; (d) measuring the colocalization of AGT with the peroxisomes in the first and second subset of cells expressing a mutant form of a peroxisomal enzyme; (e) measuring peroxisome colocalization in the images of the first and second subset of cells; and (0 determining if the candidate pharmacoperones modifies the colocalization of the mutant form of a peroxisomal enzyme, wherein if the candidate drug modifies the colocalization of the AGT to the peroxisome it is effective when compared to the placebo. In one aspect, the cells are AGT-mi and AGT-170 variants of a CHO-GO (glycolate oxidase) cell line. In another aspect, the method includes calculating a range of localization values are assigned a value ranging from −1 to 1, which is the degree of overlap of the two targets with each other independent of the intensity differences of the two targets, and is calculated using the following equation:

$${r}_{p} = \frac{\sum ( x - \dot{x} ) ( y - \dot{y} )}{\sqrt{\sum {( x - \dot{x} )}^{2} {( y - \dot{y} )}^{2}}}$$

where rₚ is the Pearson's correlation coefficient, x and y are pixel intensities of each pixel detected in the AGT and peroxisome channels, respectively, and x and y are average pixel intensities of the puncta identified as AGT and peroxisomes, respectively. In another aspect, the values are normalized on a per plate basis using the following equation:

$$\% rescue = 100 \times \frac{Test Well - Median Low Control}{Median High Control - Median Low Control}$$

wherein High Control represents the well containing AGT-mi cells treated with dimethylsulfoxide (DMSO) and Low Control represents the well containing AGT-170 cells also treated with DMSO. In another aspect, the method further comprises the step of determining cell count, nuclear intensity, morphology and condensation. In another aspect, the colocalization changes from the cytosol or mitochondria to the peroxisome. In another aspect, the mutant form of the peroxisomal enzyme of pathophysiologically relevant. In another aspect, the peroxisome in the first and second subset of cells is stained with a dye, an antibody, gold labeled antibodies, ferritin labeled antibodies, peroxidase labeled antibodies, detecting perixosomal RNA, cerium, or 3,3′-diaminobenzidine. In another aspect, the mutant form of a peroxisomal is a mutant alanine:glyoxylate aminotransferase (AGT) enzyme. In another aspect, the well is part of a multi-well plate selected from 2, 4, 6, 8, 10, 12, 24, 48, 96, 394, or 1536 well plates. In another aspect, the candidate is selected from at least one of 26-Deoxymonensin B, nigericin, salinomycin, or active derivatives thereof. In one aspect, the candidate correctly folds and/or routes otherwise-misfolded/mistrafficked mutant proteins to the correct intracellular or extracellular location.

Yet another embodiment of the present invention includes a method of determining the effectiveness of a candidate drug to treating and/or prevent protein misfolding by one or more target-specific pharmacoperones, the method comprising: (a) incubating the candidate drug to a first subset of the cells, and a placebo to a second subset of the cells; (b) fixing and staining the first and second subset of cells, wherein the stains detects anti-alanine:glyoxylate aminotransferase (AGT) enzyme in the cells; (c) generating images the first and second subset of cells with a camera; (d) measuring the co-localization of AGT with the peroxisomes in a mammalian cell based system expressing a pathophysiologically relevant mislocated mutant form of a alanine:glyoxylate aminotransferase (AGT) enzyme; and (e) determining if the candidate drug modifies the colocalization of the AGT, wherein if the candidate drug modifies the colocalization of the AGT to the peroxisome it is effective when compared to the placebo. In one aspect, the cells are AGT-mi and AGT-170 variants of a CHO-GO (glycolate oxidase) cell line. In another aspect, the method include the step of calculating a range of localization values are assigned a value ranging from −1 to 1, which is the degree of overlap of the two targets with each other independent of the intensity differences of the two targets, and is calculated using the following equation:

$${r}_{p} = \frac{\sum ( x - \dot{x} ) ( y - \dot{y} )}{\sqrt{\sum {( x - \dot{x} )}^{2} {( y - \dot{y} )}^{2}}}$$

where rₚ is the Pearson's correlation coefficient, x and y are pixel intensities of each pixel detected in the AGT and peroxisome channels, respectively, and x and y are average pixel intensities of the puncta identified as AGT and peroxisomes, respectively. In another aspect, the one or more values are obtained from the imaged cells and the values are normalized on a per plate basis using the following equation:

$$\% rescue = 100 \times \frac{Test Well - Median Low Control}{Median High Control - Median Low Control}$$

wherein High Control represents the well containing AGT-mi cells treated with dimethylsulfoxide (DMSO) and Low Control represents the well containing AGT-170 cells also treated with DMSO. In another aspect, the method further comprises the step of determining cell count, nuclear intensity, morphology and condensation. In another aspect, the colocalization changes from the cytosol or mitochondria to the peroxisome. In another aspect, the peroxisome in the first and second subset of cells is stained with a dye, an antibody, gold labeled antibodies, ferritin labeled antibodies, peroxidase labeled antibodies, detecting perixosomal RNA, cerium, or 3,3′-diaminobenzidine.

Another embodiment of the present invention includes a high throughput screen for an active agent for the treatment of comprising: plating cells comprising at least one mislocated mutant form of a peroxisomal enzyme; adding a control and compound to each plate from a library of compounds; fixing the cells; contacting the cells with an agent that detects the mislocated mutant form of a peroxisomal enzyme; and imaging the cells in the wells. In one aspect, the cells are AGT-mi and AGT-170 variants of a CHO-GO (glycolate oxidase) cell line. In another aspect, the dyes are selected to image the cells in the wells at 386, 485 and 549 nm to differentiate between localization of the mislocated mutant form of a peroxisomal enzyme to the mitochondria, peroxisome or cytosol. In another aspect, the mislocated mutant form of a peroxisomal enzyme is alanine:glyoxylate aminotransferase (AGT) enzyme. In another aspect, the mislocated mutant form of a peroxisomal enzyme is pathophysiologically relevant. In another aspect, the agent that detects the mislocated mutant form of a peroxisomal enzyme is an anti-AGT antibody. In another aspect, a membrane of the peroxisomes is detected with an anti-PMP70 antibody. In another aspect, the compound is selected from at least one of 26-Deoxymonensin B, nigericin, salinomycin, or active derivatives thereof.

Yet another embodiment of the present invention includes a high throughput screen for an active agent for the treatment of comprising: plating cells comprising at least one intracellular molecule target; adding a control and the active agent from a library of compounds to separate wells comprising the plated cells; fixing the cells; contacting the cells with an agent that detects the intracellular target; and imaging the cells in the wells, wherein a difference in the intracellular localization of the intracellular target in the cells treated with a control when compared to the active agent shows that the active agent is able to change the intracellular localization of the intracellular target molecule target. In one aspect, the intracellular target is at least one of a protein, a carbohydrate, a lipid, a nucleic acid or combinations thereof. In another aspect, the localization changes from the cytosol or mitochondria to a peroxisome. In another aspect, the active agent is selected from at least one of 26-Deoxymonensin B, nigericin, salinomycin, or active derivatives thereof. In one aspect, the active agent correctly folds and/or routes otherwise-misfolded/mistrafficked mutant proteins to the correct intracellular or extracellular location.

Yet another embodiment of the present invention includes an agent capable of changing the intracellular localization of a protein selected from at least one of 26-Deoxymonensin B, nigericin, salinomycin, or active derivatives thereof.

### DESCRIPTION OF THE DRAWINGS

For a more complete understanding of the features and advantages of the present invention, reference is now made to the detailed description of the invention along with the accompanying figures and in which:

FIG. 1 shows CHO cells were plated in 1536-well plates at 250 cells/well. After an overnight incubation time at 37 C, 95% relative humidity and 5% CO2, cells were treated with DMSO or glycerol at different concentrations. After 2 days, cells were fixed with 4% paraformaldehyde, washed 3 times with PBS and permeabilized for 10-30 min with 0.1% Triton X-100 in PBS (PBS-TX). Cells were then stained with a guinea pig anti-AGT “A1” antibody (1:10,000) and a rabbit anti-PMP70 antibody (Ab3421, 1:500) in PBS-TX containing 0.5% goat serum. After 3 PBS washes, anti-rabbit AF488 and anti-guinea pig AF546 secondary antibodies (both at 1:10,000), as well as Hoeschst at 10 ug/mL were added to each well. Images were acquired with a 20× objective on the CellInsight (Cellomics). Puncta labeled with the anti-PMP70 antibody (labeling peroxisomes) and the anti-AGT antibody are detected in the green and red channels, respectively. Colocalization of the two populations of puncta was determined via calculation of the Pearson Colocalization Coefficient using Cellomics' “Colocalization” BioApplication. Here, miAGT cells are non-mutant AGT expressing CHO cells while the mutant AGT cells are references as 170s. The concentration of glycerol used in uM is listed in both the bar graph and the scatterplot. 4 replicates per condition were done and Z′ and S:B are calculated from the untreated AGT 170 cells versus the miAGT cells. It is clear that the glycerol is stabilizing the AGT mutant.

FIGS. 2A and 2B show AGT-mi cells or AGT170 cells plated using an automated dispenser followed by the addition of DMSO via a Pintool transfer device an example of raw and normalized data from a representative DMSO plate. Five hundred CHO-K1 cells expressing either AGTmi or AGT-170 were plated in a 384-well plate and incubated overnight. The following day, all wells but the last column were treated with DMSO (0.9% final concentration) using a PinTool transfer unit. After 3 days, plates were prepared for automated, high content imaging. FIG. 2A shows the heat map of the calculated Pearson's correlation coefficient for each well of the plate, as well as the plate map. FIG. 2B is a graph using the plate map shown and the same protocol and detection algorithm described in FIG. 1, the scatterplot and plate statistics indicate the assay is robust for HCS in 1536 well format. This demonstrates the assay is compatible with liquid-handling instruments and washers available in their lab.

FIG. 3 shows a phenotypic cell based AGT co-localization 1536 well high content assay scatterplot results. 12 compound plates were screened, some in replicates which yielded satisfactory Z′, Z and S:B results. Black arrows point out 2 compounds of interest; Monensin (84% rescue) and related analog (73% rescue).

FIG. 4 shows images and RBG composite taken from 1536 well pilot screen wells including “white boxes” which are the expanded the field of view shown in the 1st column. Closer inspection of wells containing the two most active compounds demonstrates co-localization of the PMP70 and AGT labels indicating re-routing of the AGT to the peroxisomes.

FIG. 5 shows concentration response curves for 5 compounds tested that were derived from fresh powders. Monensin (SR-05000013702-3) and 26-Deoxymonensin B (SR-05000002136-1) were the top two actives from the pilot assay. SR-05000002332-2 and SR-05000002207-2 are close analogs known as nigericin and salinomycin, respectively. Fendiline, SR-01000003123-4, is inactive here and appears toxic at higher concentrations.

FIG. 6 shows the results of using the same protocol and detection algorithm described in FIG. 1, it is easy to distinguish artifact from real effect.

FIG. 7 shows a high content data management workflow diagram using, e.g., the Scripps Research Institute Molecular Screening Center (SRIMSC) database. Data can be accessed remotely upon completion of each individual plate read. The components of this system are all in place and “pressure” tested for efficacy. The integrated CellInsight HCS reader along with specialized plate handling system.

FIG. 8 is a flowchart that shows the robotic validation of the present invention.

FIG. 9 shows the distribution plots of SDDL properties.

### DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention includes a miniaturized a cell-based assay in order to identify pharmacoperone drugs present in large chemical libraries in order to selectively correct AGT misrouting. One non-limiting examples of this assay employs AGT-170, a mutant form of AGT that predominantly resides in the mitochondria, which is monitored for its relocation to the peroxisomes through automated image acquisition and analysis. Over the course of a pilot screen of 1,280 test compounds, an average Z′-factor of 0.72±0.02 was achieved, demonstrating for the first time an assay for High Throughput Screening (HTS).

As used herein, the term “pharmacoperone”, “candidate agent” and “active agent” refers to cell-permeable small molecules that serve to help correctly fold and route otherwise-misfolded/mistrafficked mutant proteins. Pharmacoperones can also be described as small molecules that rescue misfolded proteins and redirect them to their correct location thereby restoring their function and, potentially, curing disease. In one non-limiting example, G-protein-coupled receptors (GPCRs) have been heavily exploited in HTS in terms of pharmacoperone discovery with promising lead scaffolds emerging for the vasopressin 2 receptor (V2R) and the gonadotropin releasing hormone (GnRH) receptor (GnRHR). The functional rescue of these proteins by pharmacoperones has been reported by our groups and others. The discovery of target-specific pharmacoperones by high throughput screening (HTS) requires the design of automation-friendly, microtiter plate-compatible assays. The present invention includes a high throughput assay for evaluating the effectiveness of potential “pharmacoperones”, “candidate agents” or “active agents” that help correctly fold and route otherwise-misfolded/mistrafficked mutant proteins.

Previously reported assays measuring alanine:glyoxylate aminotransferase (AGT) activity are not fully compatible with HTS requirements or are effectively too far removed from the phenotypic and physiological relevance of this target. A phenotypic, microscopy-based assay is described herein that detects the co-localization of AGT with the peroxisomes in a mammalian cell based system expressing a pathophysiologically relevant mislocated mutant form of AGT; AGT-170. In the present work, we have successfully miniaturized the assay to a 384-well plate format and demonstrate its HTS-readiness through a small scale pilot screen that paves the way to interrogating larger small molecule libraries in a fully automated fashion.

Cell culture. The CHO-GO (glycolate oxidase) cell lines expressing the AGT-mi and −170 variants were described elsewhere.⁶ Cells were routinely cultured in Ham's F12 medium supplemented with 10% fetal bovine serum (Hyclone), 100 units/mL penicillin, 100 μg/mL streptomycin and 0.25 μg/mL amphotericin B (antibiotic-antimycotic mix, Gibco), as well as 400 μg/mL zeocin and 800 μg/mL G418 (Life Technologies) for selection.

Compound library. A small set of 1,280 compounds encompassing four compound plates from the library was used for the pilot screen. Details regarding Scripps' Drug Discovery collection can be found at hts.florida.scripps.edu/index.php/facilities.html#Compound-Libraries. Compounds were plated as 1 mM solutions in DMSO. The final nominal concentration during the assay cell treatment was 4 μM and the final DMSO concentration 0.4%. The skilled artisan will recognize that the library can be changed or the targets expanded for use with the present invention.

384-well plate assay protocol. One non-limiting examples of a detailed stepwise protocol is presented in Table 1. Briefly, AGT-170 cells (and AGTmi cells that serve as a positive control) were seeded in black, square, IQ™ 100 μ384-well plates (Aurora, Brooks, Chelmsford, Mass.) at 500 cells/wells using a Flying Reagent Dispenser (FRD, Beckman Coulter, Brea, Calif.). After an overnight incubation at 37° C., 95% relative humidity (RH) and 5% CO₂, cells were treated with 100 nL test compounds or DMSO for the positive and negative wells with a pintool transfer unit (GNF). Plates were then placed in an incubator for three days at 37° C., 95% RH and 5% CO₂, immunostained and acquired on a high content imager as described below.

TABLE 1
Example 384-well plate protocol for the AGT high-content assay.

| Step | Parameter | Value | | Description |
|---|---|---|---|---|
| 1 | Plate cells | 25 | μL | 500 cells per well |
| 2 | Incubation time | 16-20 | hours | 37° C., 5% CO₂ and 95% |
| | | | | relative humidity |
| 3 | Controls and library | 100 | nL | 4 μM final, 0.4% DMSO |
| | compounds | | | final |
| 4 | Incubation time | 3 | days | 37° C., 5% CO₂ and 95% |
| | | | | relative humidity |
| 5 | Dispense 2X fixation | 25 | μL | 8% Formaldehyde in |
| | solution | | | D-PBS, 4% final |
| 6 | Incubation time | 30 | min | room temperature |
| 7 | Wash | 3 | times | with D-PBS |
| 8 | Dispense | 25 | uL | 0.1% Triton-X100 in |
| | permeabilization | | | D-PBS |
| | solution | | | |
| 9 | Incubation time | 20 | min | room temperature |
| 10 | Wash | 3 | times | with D-PBS |
| 11 | Dispense primary | 8 | μL | Anti-AGT and anti-PMP70 |
| | antibody solution | | | at 1:10,000 and1:500, |
| | | | | respectively |
| 12 | Incubation time | 1 | hour | room temperature |
| 13 | Wash | 3 | times | with D-PBS |
| 14 | Dispense secondary | 8 | μL | Hoechst at 10 mg/mL and |
| | antibody solution | | | second day antibodies at |
| | | | | 1:10,000 |
| 15 | Incubation time | 1 | hour | room temperature |
| 16 | Wash | 3 | times | with D-PBS |
| 17 | Dispense D-PBS | 50 | μL | |
| 18 | Seal plate | | | |

| | | | |
|---|---|---|---|
| 19 | Image wells | 20X | record emissions at 386, |
| | | objective | 485 and 549 nm |

Immunostaining. Following treatment and incubation as described above, cells were fixed by the addition of 2× fixation solution (8% paraformaldehyde-PFA) using a FRD. Plates were then washed 3 times with PBS on the Squirt microplate washer using a 12° nozzle tilt and 15 PSI pressure according to the user's manual for washing normal binding cells in 384-well plate format (Brook, Chelmsford, Mass.). Cells were then permeabilized by dispensing a solution of 0.1% Triton X-100 in PBS (PBS-TX) and incubated for 10-30 minutes. Next, cells were stained with a guinea pig anti-AGT “A1” antibody (Danpure's Lab, London, UK; 1:10,000) and a rabbit anti-PMP70 antibody (Abcam, Cambridge, Mass.; 1:500) in PBS-TX containing 0.5% goat serum. After 3 PBS washes, anti-rabbit AF488 and anti-guinea pig AF546 secondary antibodies (Molecular Probes, Eugene, Oreg.; both at 1:10,000), as well as Hoechst at 10 μg/mL were added to each well. Thirty minutes later, plates were washed 3 times, filled with 50 μL/well of PBS and sealed with black tape.

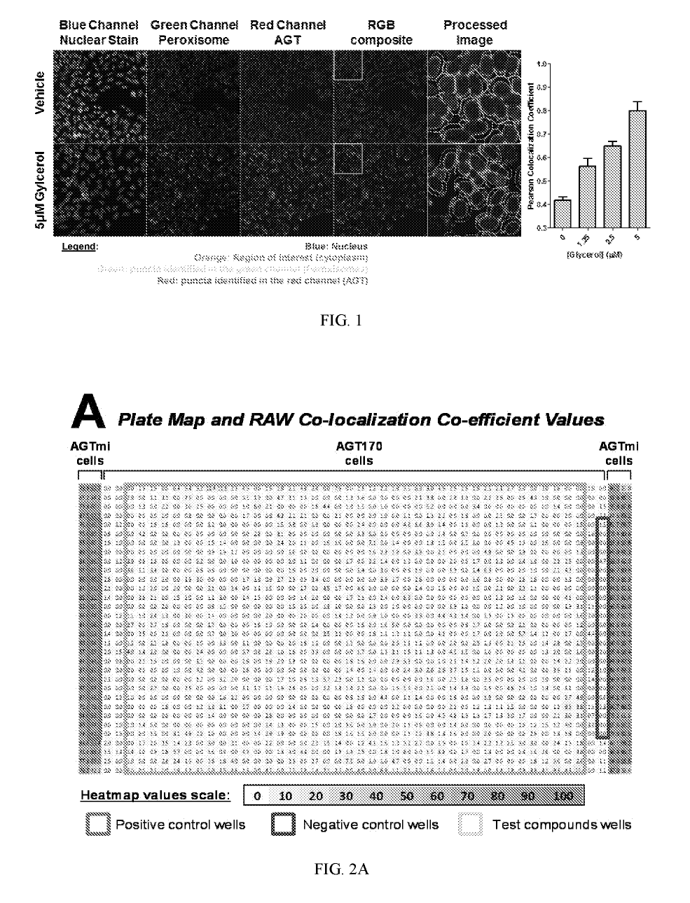

Image acquisition and analysis. Plates were read and images acquired with the CellInsight high content reader (Thermo Fisher Scientific, Pittsburgh, Pa.) using a 20× objective. A detailed and illustrated description of the image acquisition and analysis is presented FIGS. 1A to 1H. A maximum of 9 fields of view per well were acquired, yielding an average of ≈700 cells detected. The nuclear stain channel (Hoechst, 386 nm, 9.8 milliseconds exposure time) was used to focus on the cell layer. Puncta labeled with the anti-PMP70 antibody (labeling peroxisomes) and the anti-AGT antibody were acquired in the green (485 nm, 40 milliseconds exposure) and red (549 nm, 15 milliseconds exposure) channels, respectively. Nuclei were detected using the fixed threshold method with an intensity threshold of 300. Next, a cytoplasmic region of interest was created around each nucleus by creating a ring with a distance of 1 pixel from the nucleus and a width of 8 pixels. Peroxisomes and AGT proteins were identified in the cytoplasmic region of interest using a fixed intensity threshold of 200 and 141, respectively. Colocalization of the two populations of puncta was determined using the Cellomics' “Colocalization BioApplication”; the output feature providing the Pearson's correlation coefficient between the two targets is called “ROI_A(B)_Correlation Coef” This feature, that shows values ranging from −1 to 1, describes the degree of overlap of the two targets with each other independent of the intensity differences of the two targets, and is calculated using the following equation:

$${r}_{p} = \frac{\sum ( x - \dot{x} ) ( y - \dot{y} )}{\sqrt{\sum {( x - \dot{x} )}^{2} {( y - \dot{y} )}^{2}}}$$

Where rₚ is the Pearson's correlation coefficient, x and y are pixel intensities of each pixel detected in the AGT and peroxisome channels, respectively, and x and y are average pixel intensities of the puncta identified as AGT and peroxisomes, respectively.

Images and data were automatically spooled to the “STORE” database (Thermo/Cellomics) hosted on, e.g., a Scripps Research Institute Molecular Screening Center (SRIMSC) HTS server.

Data management and HTS data analysis. The relevant well feature generated by the detection algorithm, named the “MEAN_ROI_A_Correlation Coef,” was exported from the STORE database as a tabulated file using a custom Simple Object Access protocol (SOAP) web service utilizing Thermo's HCS Connect API. Reader files were then uploaded into Scripps' Drug Discovery database (Symyx Assay Explorer, Santa Clara, Calif.). Plate Z-factor, Z′-factor and Sample to Background ratio (S/B) were automatically calculated as previously described. Assay results were normalized on a per plate basis using the following equation:

$$\% rescue = 100 \times \frac{Test Well - Median Low Control}{Median High Control - Median Low Control}$$

where High Control represents the well containing AGTmi cells treated with DMSO (n=32) and Low Control represents the well containing AGT-170 cells also treated with DMSO (n=16).

Development of a HTS-compatible, microscopy-based assay to monitor AGT-peroxisome colocalization. Stably transformed CHO cells were used to study the subcellular distribution of normal and various PH1 mutant AGT constructs by immunofluorescence microscopy. Among these mutants, the AGT-170 variant presented the most severe peroxisome-to-mitochondrion mistargeting phenotype. Accordingly, a cell line expressing this specific mutation was used to design a high-content, high-throughput assay to identify potential pharmacoperones able to rescue AGT mistargeting by rerouting it to the peroxisomes. In order to be compliant with HTS requirements, the original assay had to be simplified, adapted to microtiter plates and automated.

1536-well plate assay protocol. An example of the 1536-well methods and miniaturized is illustrated in FIG. 1, showing the staining of AGT and mitochondria in the cell lines. The statistical data for each well shows the Pearson Correlation coefficient. The data shown demonstrate the basis of an high throughput screen (HTS) functional screen since AGT-mi is mainly located in the peroxisome while AGT170 is located in the mitochondria. The AGT 152 cells show even closer co-localization with the mitochondrion marker, compared to AGT170. The ability to detect and measure AGT/peroxisome colocalization was determined with the HTS-compatible, wide-field high content imager by labeling CHO cells expressing either the AGT-170 PH1 mutant or the AGT minor allele for nuclei, peroxisomes and AGT in 96-well plate format. Acquisition was made using a 20× objective. A dedicated detection algorithm was created that automatically calculates and reports the Pearson's colocalization coefficient between the AGT- and peroxisome-labeled areas for each well (FIG. 1).

FIG. 1 shows CHO cells plated in 1536-well plates at 250 cells/well. After an overnight incubation time at 37 C, 95% relative humidity and 5% CO₂, cells were treated with DMSO or glycerol at different concentrations. After 2 days, cells were fixed with 4% paraformaldehyde, washed 3 times with PBS and permeabilized for 10-30 min with 0.1% Triton X-100 in PBS (PBS-TX). Cells were then stained with a guinea pig anti-AGT “A1” antibody (1:10,000) and a rabbit anti-PMP70 antibody (Ab3421, 1:500) in PBS-TX containing 0.5% goat serum. After 3 PBS washes, anti-rabbit AF488 and anti-guinea pig AF546 secondary antibodies (both at 1:10,000), as well as Hoeschst at 10 ug/mL were added to each well. Images were acquired with a 20× objective on the CellInsight (Cellomics). Puncta labeled with the anti-PMP70 antibody (labeling peroxisomes) and the anti-AGT antibody are detected in the green and red channels, respectively. Colocalization of the two populations of puncta was determined via calculation of the Pearson Colocalization Coefficient using Cellomics' “Colocalization” BioApplication. Here, miAGT cells are non-mutant AGT expressing CHO cells while the mutant AGT cells are references as 170s. The concentration of glycerol used in uM is listed in both the bar graph and the scatterplot. 4 replicates per condition were done and Z′ and S:B are calculated from the untreated AGT 170 cells versus the miAGT cells. The glycerol is stabilizing the AGT mutant.

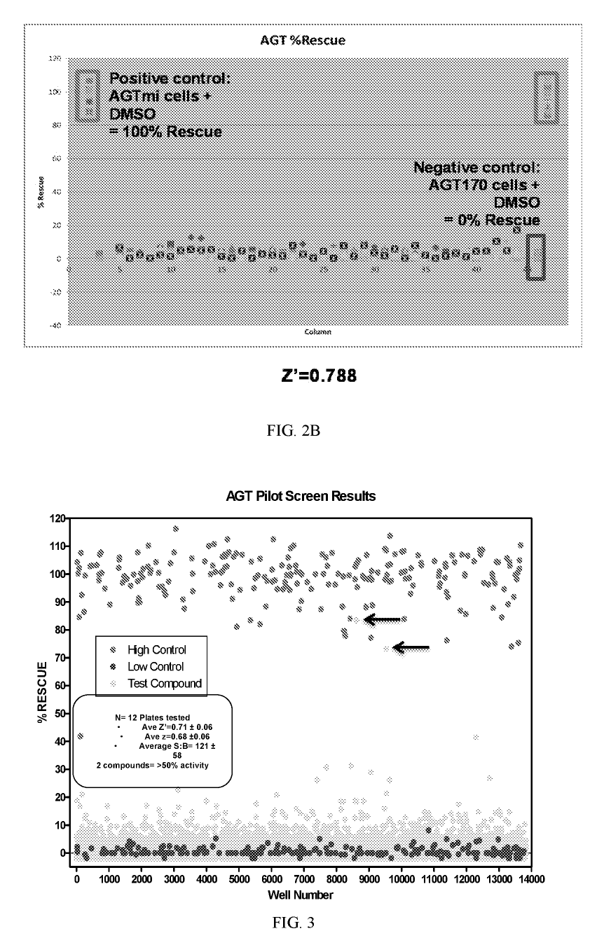

This 1536 well assay was further validated and demonstrates readiness for HTS using a whole plate assay. The results as shown in FIGS. 2A and 2B indicate robust statistics that are amenable to HCS. In FIG. 2A, either AGT-mi cells or AGT170 cells were plated using an automated dispenser followed by the addition of DMSO via a Pintool transfer device. In FIG. 2B, using the plate map shown and the same protocol and detection algorithm described in FIG. 1, the scatterplot and plate statistics indicate the assay is robust for HCS in 1536 well format. This demonstrates the use of the assay and is shown to be compatible with liquid-handling instruments and washers.

In order to assess the ability to induce and detect an increase in the Pearson's colocalization coefficient, both AGTmi and AGT-170 cells were treated with glycerol, which is a viscous polyol compound known to enhance the stability of proteins in solution. This co-solvent shifts the native protein to more compact states and inhibits protein-protein aggregation while proteins are being refolded during biological synthesis. Accordingly, treatment with increasing concentrations of glycerol resulted in a marked increase of Pearson's colocalization coefficients for the AGT-170 cells changing from 0.41±0.01 to 0.80±0.04 (n=4); i.e. a 1.91 fold increase (data not shown). Notably, AGTmi cells were also showing an increase in Pearson's values upon glycerol treatment, albeit this increase was marginal (˜1.10 fold increase). Concentrations of glycerol higher than 5% affected cell viability and prevented accurate determination of the Pearson's colocalization coefficient; for these reasons, they were avoided. To probe the robustness of the assay, the glycerol titration assay was repeated on two separate plates. Pearson's coefficient determined from multiple wells treated with varying glycerol concentrations ranging from ≈0.4 to ≈0.9 and indicates that data generated from the two separate plates were virtually identical, yielding a R² greater than 0.99. The Z′-factor, a statistical measurement indicative of HTS-readiness, was calculated between the AGT-170 cells in absence and presence of 5% glycerol and found to be 0.58, and at 0.81 between untreated AGT-170 and AGTmi cells.

Miniaturizing and automating the AGT-170 cell-based assay to the 384-well plate format. With Z′-factor values greater than 0.5, the assay was further miniaturized and automation to the 384-well plate format. Whereas the majority of HTS assays that are homogeneous in format typically rely on three dispense steps (cell addition, compound delivery, detection reagents dispense), immunostaining-based assay protocols often require minimum of eight dispenses, including aspiration and/or wash steps. A primary concern was that automated liquid handling devices would disrupt the cell layer during this procedure. To prevent this from occurring, the cells were fixed as early as possible in the protocol by replacing the initial media aspiration step with a dispense of a 2× concentrated fixation solution. In addition, a non-contact plate washer was used that does not rely on aspiration to empty the wells, but instead utilizes an “air blade” that ejects liquids from the well with little to no residual volume (Squirt, Brooks, Chelmsford, Mass.). To verify that cells remained in the wells and were not being dislodged during the plate preparation process, Hoechst staining was added during the fixation step of a mock immunostaining protocol and nuclei were counted at each step. The dispensing and washing conditions were optimized with little to no cell detachment over the course of the plate preparation process (data not shown). These results also indicated that cells seeded at densities higher than 600 cells per well reached total confluence; a cell seeding density of 500 cells per well was considered optimal and used for the rest of this study. Volumes, concentration and incubation times were optimized to offer the best balance between cost per well, time and assay performance. One example of the resulting miniaturized assay protocol is presented in Table 1.

Validation for HTS. To verify that the assay was compatible with HTS requirements, the potential for position effects by running plates treated with DMSO only, was investigated. A heat map of a representative plate treated with DMSO is shown FIGS. 2A and 2B; this plate did not show any edge effect or position effect within the sample field, indicating that despite an extended incubation time (4 days), all variables that can lead to well-to-well variability (such as temperature gradient, evaporation, contamination, etc.) were tightly controlled. In FIG. 2A, either AGT-mi cells or AGT170 cells were plated using an automated dispenser followed by the addition of DMSO via a Pintool transfer device. In FIG. 2B, using the plate map shown and the same protocol and detection algorithm described in FIG. 1, the scatterplot and plate statistics indicate the assay is robust for HCS in 1536 well format. The coefficients of variation (CVs) of 6.12%, 1.17% and 7.47% for the sample field, high control and low control, respectively, were well under an empirically accepted maximum of 10%. The Z′-factor was 0.62, demonstrating the miniaturized AGT assay's robustness and readiness for drug screening purposes. Also the lack of observable outliers within the sample field in general indicates this assay should be fairly devoid of false positives or negatives. The assay does have at least some indication of DMSO sensitivity as observed in column 24 of FIGS. 2A and 2B with the normalized data displaying a negative % rescue for non-treated wells.

Screen. In order to assess the performance of the AGT assay under HTS conditions, a pilot screen against a set of 1,280 diversified molecules was conducted. The final concentration at which the compounds were tested was 4 μM, which corresponds to a final DMSO concentration of 0.4%. A total of six plates were used for this assay, comprising four separate compounds plates and two DMSO plates, one at the beginning and at the end of the run. The sample to background ratio (S/B), Z- and Z′-factors of each plate. FIG. 3 shows the results from a phenotypic cell based AGT co-localization 1536 well high content assay scatterplot results. 12 compound plates were screened, some in replicates which yielded satisfactory Z′, Z and S:B results. Black arrows point out 2 compounds of interest; Monensin (84% rescue) and related analog (73% rescue).

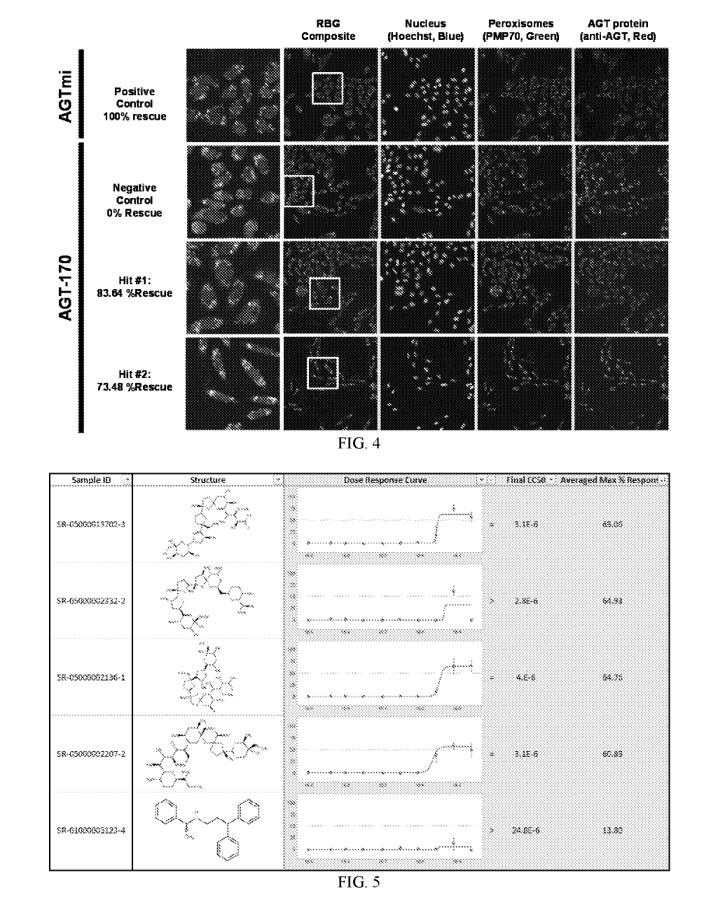

With this assay performing well as demonstrated by Z′, Z, signal to basal, as monitored and normalized to the mutant vs. WT cells, a 4.6K pilot screen including LOPAC 1280, an FDA approved library (˜3200 compounds) as well as the NCI oncologic drug set consisting of 114 compounds was conducted. All data was imported into the Scripps database and quality controlled (QC′d) prior to analysis. The outcome was significant in terms of not only assay robustness but importantly we identified two hits with appreciable activity greater than 50% (FIG. 3). As an example, a 50% cut-off was used due to the limited number of compounds tested. Setting the bar at a more typical HTS cut-off such as 3 standard deviations plus average led to some noise; however additional modifications to the methods can be used to improve cut-off determination. High content analysis affords the user of deep interrogation of wells to cells type analysis. Technologically this provides an advantage over other HTS formats which, upon further inspection of the HCS images one can readily see that, compared to controls, the two hits indeed appear to rescue the WT phenotype (FIG. 4). FIG. 4 shows images and RBG composite taken from 1536 well pilot screen wells including “white boxes” which are the expanded the field of view shown in the 1st column. Closer inspection of wells containing the two most active compounds demonstrates co-localization of the PMP70 and AGT labels indicating re-routing of the AGT to the peroxisomes.

The new design, miniaturization and validation of a cell-based HTS assay as described was used to enable the monitoring of re-routing of the AGT-170 mutant protein to its correct location, the peroxisomes. An automated, high content microscopy can be considered a technology of choice to accurately measure AGT re-routing; it was indeed favored over other technologies relying on reporter systems that usually require creating fusion or tagged proteins and can potentially lead to assay artifacts. In contrast, using immunodetection allowed the use of AGT proteins that are devoid of any modifications and hence represent exact carbon copies of those found in patients.

Surprisingly, it appears that the use of a wide-field microscope yields enough spatial resolution to be able to clearly map AGT location with regards to the peroxisomes. Even previous work relied on four acquisition channels (nuclear stain, anti-AGT and peroxisomes immunostaining, and MitoTracker Red), it was reasoned that for HTS purposes the MitoTracker channel could be removed, since it is now well documented that the AGT-170 mutant protein is predominantly located in the mitochondria. The goal is indeed to confirm its rerouting to the peroxisome upon treatment with a hit compound rather than its departure from the mitochondria.

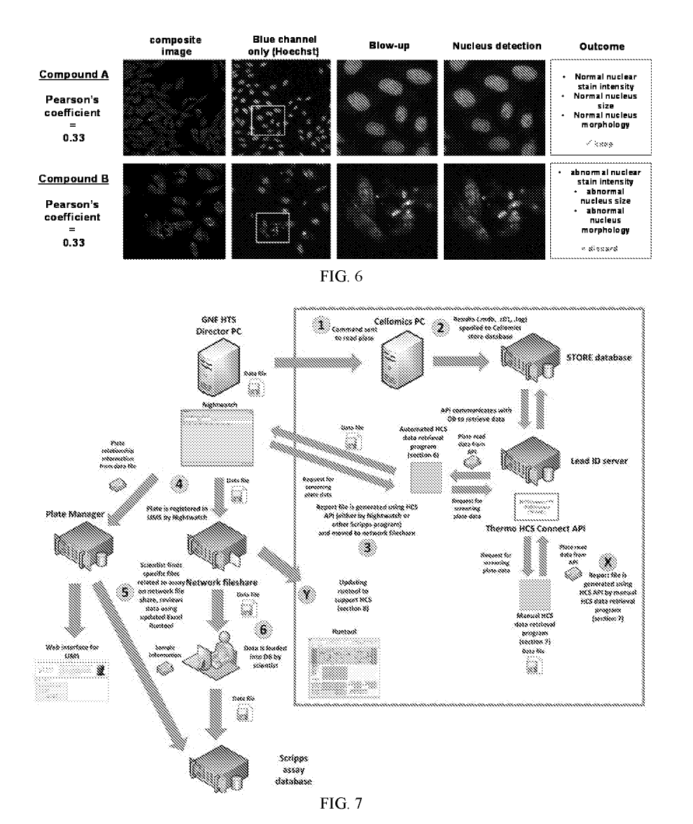

To show the reliability and reproducibility of this assay, top active analogs, two additional chemically related analogs as well as fendiline, were used to test the next most active compound using the assay. Fresh powders, solvated in DMSO, were obtained and reformatted as ten point three fold serial dilutions and proceeded to test them in the same assay as described above. The assay performed well, indicative of its day-to-day reproducibility. All data was imported into the Scripps database, assessed for quality control in terms of Z′, S:B and CV (i.e., quality controlled), and curves were fitted using a 4-parameter algorithm without constraining the top or bottom asymptote. An EC₅₀ was determined for each curve by plotting concentration versus normalized activity (25 uM was the highest concentration tested, however, higher concentrations can be tested using the present invention). The concentration response curves are displayed in FIG. 5 and demonstrate the monesin (SR-05000013702-3) and analogs reproduce the activity found in the pilot screen and generate low micromolar potency. Fendiline did not show significant activity but upon further interrogation appeared toxic at 10 uM by eye when observing HCS images (FIG. 6).

In addition to the biology of interest, and without any additional plate preparation, high content imaging offers the opportunity to capture additional cell features, such as cell count, nuclear intensity, morphology and condensation, which can help identify undesirable cytotoxic compounds early during the screening process.

As mentioned, high content imaging also provides an opportunity to eliminate artifacts and abnormalities associated with compounds by using additional cell features acquired during the same read process. For each well, cell number, nuclear morphology and staining intensity of the nucleus can be determined using the Hoechst channel only (FIG. 6). Cytotoxic compounds can then be easily identified and removed from further analysis. In addition, potential fluorescent compounds can be detected by carefully monitoring the fluorescence levels of the different channels.

High content assays are difficult due to the sheer volume (terabytes) of data output due in part by the ability to measure multiple complex phenotypic and generate dozens of outputs per well to the end user. The method was adapted and its data management work flow as shown in FIG. 7 addresses the computational challenge and has enabled a fully integrated robotically compatible HCS system. Briefly, at step 1, a command is sent to read the plate, with the resulting database of information processed at step 2, e.g., to a cellnomics computer or processor. The results, at step 2, can be in any number of database formats (e.g., .mdb, .c01, .log, etc.), which can be spooled to a store database, such as a cellnomics store database. The STORE database connects to a Lead ID server, which can be connected to an Automated HCS data retrieval program or a Thermo HCS Connect API, which includes generating reports from a manual HCS data retrieval (at step X) or via an automated HCS data retrieval program, with a report file being generated. The Automated HCS retrieval program can transfer a data file to and from the graphical user interface (GUI), which program can also generate a report file using the HCS API or other program, which can also share the files via a network file share at step 3. The graphic user interface can connect, at step 4, with a plate manager that records the plate relationship information from the data file. Alternatively, the data file is registered by the GUI into a network file share, which then sends sample information to a terminal used by a user at step 6, or send the information at step y, which updates the runtool to support the HCS. The plate manager can also send file information, at step 5 to, e.g., an assay database, such as the Scripps Assay Database used to demonstrate the present invention. At step 5, the GUI provides the user with specific files relating to the assay on a network file share, permits display and review of the data by the user, and can also use an updated Excel Runtool.

Taken together the AGT HCS assay has been optimized for conditions that should ensure its success in HTS. Controls such as mutant and wild type cells will be used but we now have monesin as a putative specific small molecule control. The assay can optimized and characterized as a HCS assay to help triage and/or evaluate cytotoxic compounds using doxorubicin as a control; a known toxicity agent. As shows herein, these assays have been fully validated in 384 and 1536 well format in terms of their performance associated to Z′, S:B and day-to-day reproducibility.

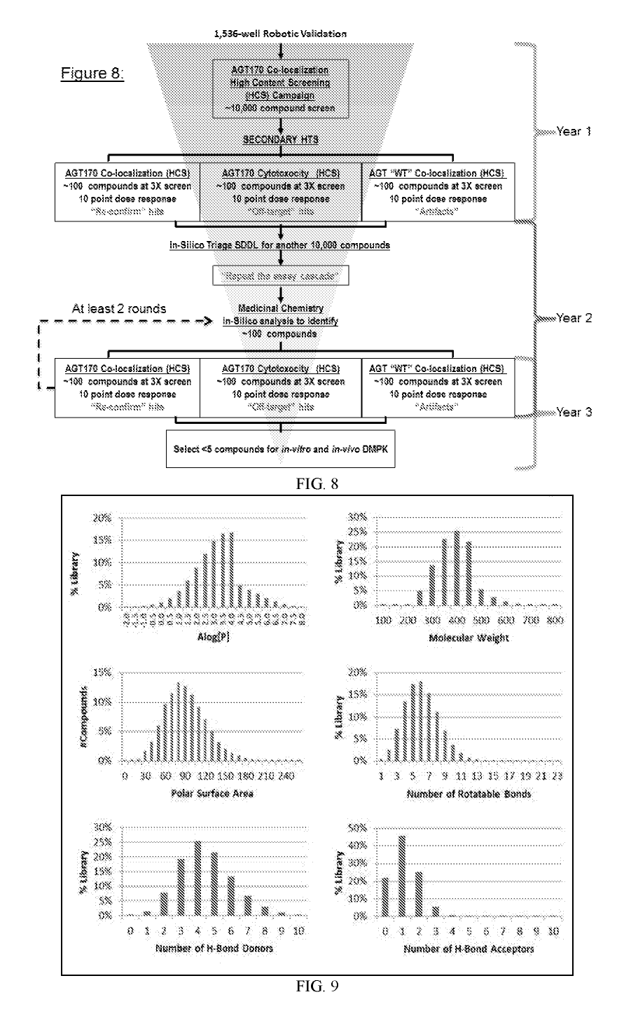

HTS and HCS assays can be used with a robot to assess stability and compatibility with our system. Based on the robotic validation and implementation of 1536 well formats, batches of ˜10K compounds; each round being progressively more focused than the first, can be tested. This is accomplished via utilizing in-silico tools for clustering and promiscuity analysis. The most promising candidate compounds can be identified and the SDDL re-visited to purposefully include compounds surrounding this rational. An exemplary screening cascade is shown in FIG. 8. Briefly, FIG. 8 shows an example with a 1,536 well plate robotic validation, in which AGT170 co-localization High Content Screening (HCS) campaign of approximately 10,000 compounds are screened. A Secondary High Throughput Screen is then conducted to differentiate between hits that are re-confirmed, off-target hits, and artifacts. Next, an in-silico triage SDDL for another 10,000 compounds is conducted. This is the repeat assay cascade. At this step, medicinal chemistry is conducted, e.g., in-silico, to identify approximately 100 compounds. These 100 compounds are again run for the detection of hits that are re-confirmed, off-target hits, and artifacts. The last step is repeated at least two more times to finally select approximately 5 or less compounds for further in vitro and in vivo DMPK testing. Certain time periods are provided along side the flowchart, which are provided solely as a guideline and not a limitation of the present invention. The skilled artisan will recognize that the process can be expanded or contracted depending on the total number of available compounds, the specificity of the results (consistent re-confirmed hits, versus some ambiguity in the results), the extent of medicinal chemistry involved and the ready availability of related compounds, and the number of devices used for the screening steps.

Example: Screening of Scripps Drug Discovery Library (SDDL). The Scripps Drug Discovery Library (SDDL) includes 644,951 compounds, representing a diversity of drug-like compound scaffolds targeted to traditional and non-traditional drug-discovery biological targets. The SDDL has been curated from over 20 commercial and academic sources and contains more than 20,000 compounds unique to Scripps. It is important to note that the SDDL also has minimum overlap (10%) with the NIH's MLPCN/MLSMR compound library, any one of which can be used with the present invention. The SDDL compounds are selected based on scaffold novelty, physical properties and spatial connectivity. A summary of select SDDL properties is shown in FIG. 9.

By design, the diversity of the SDDL mimics that of much larger collections found at major pharmaceutical companies, yet is responsive to lessons learned from successful drug discovery efforts and emerging trends in HTS library construction. The SDDL continues to be augmented with diverse small molecule scaffolds, as well as focused sub-libraries targeted to popular drug-discovery targets and compound collections provided by Scripps' distinguished chemistry faculty.

In its current state, the SDDL has several focused sub-libraries for screening popular drug-discovery target classes (e.g. kinases/transferases, GPCRs, ion channels, nuclear receptors, hydrolases, transporters), as well as diverse chemistries (e.g. click-chemistry, PAINS-free, Fsp3 enriched, and natural product collections) and physical properties (“rule-of-five,” “rule-of-three,” polar surface area, etc.). All of these can be used with the present invention, alone or in combination.

Oxalosis Chemistry/Drug Kinetics and Pharmacokinetic (DMPK) Studies. Screening hits can be prioritized based upon selectivity and potency, after hit activity is confirmed through purchase of an authentic sample (or by internal synthesis). Confirmed hits can be computationally assessed using structural similarity algorithms to identify SAR relationships that are made apparent from the uHTS data, such as active/inactive and selective/non-selective analogs. Hits registered in PubChem or previously screened in the SDDL will be checked for off-target activity to eliminate from consideration nuisance compounds/frequent hitters. Ease of analog synthesis and chemical tractability (including evaluation of parameters such as H-bond donor/acceptor count, polar surface area, cLogP, chemical stability, and absence of toxicity-associated groups) can also be used to select preferred hits for follow-up studies using the present invention. Hit scaffolds with instability issues or toxicology structure alerts will not be pursued unless chemistry strategies exist to quickly address those concerns. Confirmed tractable hits can be further developed through a battery of biochemical and cell-based assays to identify at least 3-4 compounds or chemical series that meet or exceed lead criteria, as outlined in the chemical probe development plan.

In vitro and in vivo studies can be used to determine the metabolic stability of candidate drug-like compounds as well as predict metabolic interactions and issues with biological toxicity can be conducted. The top leads from each round of SARs can progress to be evaluated in a battery of in vitro and in vivo DMPK studies (e.g., stability to rodent and human liver microsomes, CYP450 inhibition, aqueous solubility, and PAMPA or Caco-2 permeability). The present invention can be used to improve the potency and selectivity while striking a balance with obtaining acceptable DMPK properties. The DMPK Core will also perform rodent studies for PK properties of top leads, to determine peak plasma concentration (Cmax), oral bioavailability, exposure (AUC), half-life (t1/2), clearance (CL), and volume of distribution (Vd). At this stage we anticipate having several leads suitable to be advanced as probes that are acceptable for future AGXT 170 in vivo animal model studies.

Dihydrofolate reductase (DHFR)-based misfolding assay. This assay depends on AGT inserted in the middle of a reporter protein, dihydrofolate reductase and expressed in yeast lacking this essential reductase. Data suggest that a decrease in stability of AGT results in a decrease in stability/activity of the DHFR reporter required for yeast growth. Decreased stability of AGT is reflected in a reduction in yeast growth, providing the basis of a screen. For the reasons noted above, we feel a mammalian HTS model is preferable.

Differential scanning fluorometry (DSF) measures protein stability in a solution and ligand-induced changes in protein stability. The method is easily adaptable to a high-throughput format and can be carried out using a conventional real-time PCR machine. DFS has a disadvantage for screens in that it only measures functional stability rather than trafficking or other cellular events. Prior to the present invention, this technique was limited in numbers of compounds that could be screened and cannot address very large libraries. Moreover, working on proteins in solution does not enable measurement of intracellular trafficking.

Thus, HTS can be used with high content AGT co-localization assays against at least 2 rounds of 10K compounds. This led the discovery and understanding of this novel therapeutic approach and previously designed GLP assays that have been successfully used and led to approval of drugs for human use.

The data shown herein indicate that available materials can be used for an HTS. The assays are robust, reproducible, and have a readout that is amenable to automated analysis that can been miniaturized to a 1536-well format or smaller.

The present invention includes a robotic validation and compatibility of the high content assay for automated screening. The invention has been used for the completion of two rounds of iterative screening of 10K compounds from the SDDL. In-silico chemistry analysis was used that can include clustering, PAINS analysis, promiscuity etc. Medicinal and analytical chemistry optimization/support following the completion of the HTS phase on roughly 100 analogs can then be followed by at two more rounds of triage and more analogs being supplied albeit at a decreased number (˜50) per round.

Other mutants: Once “hits” are identified other mutants associated with the human disease state to determine can be used to determine whether these hits correct trafficking of multiple mutants, as has been observed in the case of both the V2R and the GnRHR. This shows that pharmacoperones stabilize a nucleus of the protein that is essential for stability. This observation extends the therapeutic reach of these drugs.

In vivo model: A humanized transgenic knockout mouse model (developed by Dr. Eduardo Salido at the University of La Laguna, Spain) can be used with the present invention. This mouse is hyperoxaluric due to the absence of Agxt1 expression due to misrouting of this enzyme. They have also introduced the most common mutations of the minor haplotype, G170R and I244T, into transgenic mice, and crossed these into the AgtKO line. These animals can be useful to provide an in vivo test for promising hits developed from the high-throughput screens.

As such, a GPCR mutant can be rescued with pharmacoperone drugs in vivo in a different knock-in mouse. A previous pharmacoperone drug was also an antagonist (all pharmacoperones of the GnRHR known at that time were selected from antagonist screens). Accordingly, catheterization of the left carotid and a dual pump system was needed to deliver pulses of the drugs to the pituitary, then wash it out after rescue occurred. Enzymatic assays for AGT to exclude antagonists from consideration can also be used. Accordingly elaborate surgical procedures are not required.

Rescue of the mutant enzyme in liver samples can be determined. The activity of the peroxisomal enzyme alanine:glyoxylate aminotransferase (AGT, EC 2.6.1.44) can also be measured. Once the hits are identified, those that are known to be toxic to mice (or enzyme antagonists) are eliminated and doses and frequency of administration of the drugs based on serum half lives of the drugs can be determined.

Monensin, which is used to characterize the assay, can be used to evaluate candidates in animals, since it is widely used as a feed additive and toxicity occurs only at very high doses. Mice fed diets containing 0, 37.5, 75, 150 or 300 ppm monensin for 3 months had modestly reduced body weight gain in all test groups but no other physical signs.

Monensin can be used oral gavage for administration. When this is not possible for other drugs (i.e., low oral bioavailability) these can be administer through the vena cava or one of the branches since those can be “tied off” around the catheter. Post-injection bleeding after the penetration of these large vessels can be a problem but can be controlled with glue and mesh patches.

In conclusion, a novel, cost-effective and robust miniaturized high-content assay for the discovery of pharmacoperones that can rescue an enzyme-trafficking defect involved in primary hyperoxaluria 1 is demonstrated herein. The protocol designed yielded satisfactory assay statistics and demonstrated its compatibility with HTS requirements. Integration of additional well features that can help annotate the potential deleterious effect of test compounds and further miniaturization to the 1,536-well plate format can be added. The assay presented herein provides for the first time a large library robotic screen and/or platform to identify pharmacoperones able to rescue, e.g., AGT-170 mistrafficking. The assay can also easily be adapted to other mistrafficked proteins regardless of their enzymatic activity. Finally, in addition to screening drug libraries, this assay can also be used to uncover genes and proteins involved in AGT trafficking regulation by interrogating cDNA or siRNA libraries.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Conn, P. M., et al., Transitioning pharmacoperones to therapeutic use: in vivo proof-of-principle and design of high throughput screens. Pharmacol Res, 2014. 83: p. 38-51.
2. Conn, P. M., A. Ulloa-Aguirre, and J. A. Janovick, “Pharmacoperone”: what's in a word? Pharmacol Res, 2014. 83: p. 1-2.
3. Tao, Y. X. and P. M. Conn, Chaperoning G protein-coupled receptors: from cell biology to therapeutics. Endocr Rev, 2014. 35(4): p. 602-47.
4. Janovick, J. A., et al., Restoration of testis function in hypogonadotropic hypogonadal mice harboring a misfolded GnRHR mutant by pharmacoperone drug therapy. Proc Natl Acad Sci USA, 2013. 110(52): p. 21030-5.
5. Danpure, C. J., Molecular etiology of primary hyperoxaluria type 1: new directions for treatment. Am J Nephrol, 2005. 25(3): p. 303-10.
6. Danpure, C. J., Primary hyperoxaluria type 1: AGT mistargeting highlights the fundamental differences between the peroxisomal and mitochondrial protein import pathways. Biochim Biophys Acta, 2006. 1763(12): p. 1776-84.
7. Williams, E. L., et al., Primary hyperoxaluria type 1: update and additional mutation analysis of the AGXT gene. Hum Mutat, 2009. 30(6): p. 910-7.
8. Purdue, P. E., Y. Takada, and C. J. Danpure, Identification of mutations associated with peroxisome-to-mitochondrion mistargeting of alanine/glyoxylate aminotransferase in primary hyperoxaluria type 1. J Cell Biol, 1990. 111(6 Pt 1): p. 2341-51.
9. Danpure, C. J., et al., Enzymological and mutational analysis of a complex primary hyperoxaluria type 1 phenotype involving alanine:glyoxylate aminotransferase peroxisome-to-mitochondrion mistargeting and intraperoxisomal aggregation. Am J Hum Genet, 1993. 53(2): p. 417-32.
10. Amoroso, A., et al., AGXT gene mutations and their influence on clinical heterogeneity of type 1 primary hyperoxaluria. J Am Soc Nephrol, 2001. 12(10): p. 2072-9.
11. Coulter-Mackie, M. B., et al., Three novel deletions in the alanine:glyoxylate aminotransferase gene of three patients with type 1 hyperoxaluria. Mol Genet Metab, 2001. 74(3): p. 314-21.
12. van Woerden, C. S., et al., Clinical implications of mutation analysis in primary hyperoxaluria type 1. Kidney Int, 2004. 66(2): p. 746-52.
13. Janovick, J. A., et al., Structure-activity relations of successful pharmacologic chaperones for rescue of naturally occurring and manufactured mutants of the gonadotropin-releasing hormone receptor. J Pharmacol Exp Ther, 2003. 305(2): p. 608-14.
14. Morello, J. P., et al., Pharmacological chaperones rescue cell-surface expression and function of misfolded V2 vasopressin receptor mutants. J Clin Invest, 2000. 105(7): p. 887-95.
15. Conn, P. M., et al., G protein-coupled receptor trafficking in health and disease: lessons learned to prepare for therapeutic mutant rescue in vivo. Pharmacol Rev, 2007. 59(3): p. 225-50.
16. Janovick, J. A., et al., Refolding of misfolded mutant GPCR: post-translational pharmacoperone action in vitro. Mol Cell Endocrinol, 2007. 272(1-2): p. 77-85.
17. Purdue, P. E., et al., Characterization and chromosomal mapping of a genomic clone encoding human alanine:glyoxylate aminotransferase. Genomics, 1991. 10(1): p. 34-42.
18. Leiper, J. M., P. B. Oatey, and C. J. Danpure, Inhibition of alanine:glyoxylate aminotransferase 1 dimerization is a prerequisite for its peroxisome-to-mitochondrion mistargeting in primary hyperoxaluria type 1. J Cell Biol, 1996. 135(4): p. 939-51.
19. Lumb, M. J., G. M. Birdsey, and C. J. Danpure, Correction of an enzyme trafficking defect in hereditary kidney stone disease in vitro. Biochem J, 2003. 374(Pt 1): p. 79-87.
20. Coulter-Mackie, M. B. and Q. Lian, Partial trypsin digestion as an indicator of misfolding of mutant alanine:glyoxylate aminotransferase and chaperone effects of specific ligands. Study of a spectrum of missense mutants. Mol Genet Metab, 2008. 94(3): p. 368-74.
21. Behnam, J. T., et al., Reconstruction of human hepatocyte glyoxylate metabolic pathways in stably transformed Chinese-hamster ovary cells. Biochem J, 2006. 394(Pt 2): p. 409-16.
22. Mesa-Tones, N., et al., The role of protein denaturation energetics and molecular chaperones in the aggregation and mistargeting of mutants causing primary hyperoxaluria type I. PLoS One, 2013. 8(8): p. e71963.
23. Madoux, F., et al., Development of a phenotypic high-content assay to identify pharmacoperone drugs for the treatment of primary hyperoxaluria type 1 by high-throughput screening. Assay Drug Dev Technol, 2015. 13(1): p. 16-24.
24. Breier, J. M., et al., Development of a high-throughput screening assay for chemical effects on proliferation and viability of immortalized human neural progenitor cells. Toxicol Sci, 2008. 105(1): p. 119-33.
25. Berlinicke, C. A., et al., High-content screening data management for drug discovery in a small- to medium-size laboratory: results of a collaborative pilot study focused on user expectations as indicators of effectiveness. J Lab Autom, 2012. 17(4): p. 255-65.
26. Zhang, J. H., T. D. Chung, and K. R. Oldenburg, A Simple Statistical Parameter for Use in Evaluation and Validation of High Throughput Screening Assays. J Biomol Screen, 1999. 4(2): p. 67-73.
27. Leeson, P. D. and B. Springthorpe, The influence of drug-like concepts on decision-making in medicinal chemistry. Nat Rev Drug Discov, 2007. 6(11): p. 881-90.
28. Axerio-Cilies, P., et al., Investigation of the incidence of “undesirable” molecular moieties for high-throughput screening compound libraries in marketed drug compounds. Eur J Med Chem, 2009. 44(3): p. 1128-34.
29. Harvey, A. L., Natural products in drug discovery. Drug Discov Today, 2008. 13(19-20): p. 894-901.
30. Potashman, M. H. and M. E. Duggan, Covalent modifiers: an orthogonal approach to drug design. J Med Chem, 2009. 52(5): p. 1231-46.
31. Baell, J. B. and G. A. Holloway, New substructure filters for removal of pan assay interference compounds (PAINS) from screening libraries and for their exclusion in bioassays. J Med Chem, 2010. 53(7): p. 2719-40.
32. Bialkowska, A. B., et al., Identification of small-molecule inhibitors of the colorectal cancer oncogene Kruppel-like factor 5 expression by ultrahigh-throughput screening. Mol Cancer Ther, 2011. 10(11): p. 2043-51.
33. Amundsen, S. K., et al., Small-molecule inhibitors of bacterial AddAB and RecBCD helicase-nuclease DNA repair enzymes. ACS Chem Biol, 2012. 7(5): p. 879-91.
34. Lipinski, C. A., et al., Experimental and computational approaches to estimate solubility and permeability in drug discovery and development settings. Adv Drug Deliv Rev, 2001. 46(1-3): p. 3-26.
35. Walters, W. P., Going further than Lipinski's rule in drug design. Expert Opin Drug Discov, 2012. 7(2): p. 99-107.
36. Rishton, G. M., Nonleadlikeness and leadlikeness in biochemical screening. Drug Discov Today, 2003. 8(2): p. 86-96.
37. Hopper, E. D., et al., In vivo and in vitro examination of stability of primary hyperoxaluria-associated human alanine:glyoxylate aminotransferase. J Biol Chem, 2008. 283(45): p. 30493-502.

1. A method of determining the effectiveness of one or more drug candidates to change the intracellular localization of a target molecule, the method comprising:
(a) incubating the one or more drug candidates with a first subset of the cells, and a control agent with a second subset of the cells;
(b) fixing and staining the first and second subset of cells, wherein the stain detects the target molecule;
(c) generating images of the first and second subset of cells with a camera;
(d) measuring the difference in the intracellular localization of the target molecule in the first as compared to a second subset of cells; and
(e) determining if the drug candidate modifies the localization of the intracellular localization of the target protein, wherein if the candidate drug modifies the intracellular localization of the target protein when compared to the placebo it is an effective drug candidate.

2. The method of claim 1, wherein a range of localization values are assigned a value ranging from −1 to 1, which is the degree of overlap of the two targets with each other independent of the intensity differences of the two targets, and is calculated using the following equation:
$${r}_{p} = \frac{\sum ( x - \dot{x} ) ( y - \dot{y} )}{\sqrt{\sum {( x - \dot{x} )}^{2} {( y - \dot{y} )}^{2}}}$$
Where rₚ is the Pearson's correlation coefficient, x and y are pixel intensities of each pixel detected for the target protein in the first versus the second subset of cells, respectively, and x and y are average pixel intensities of the puncta identified as a position of the target protein in the first versus the second subset of cells, respectively.

3. The method of claim 2, wherein the values are normalized on a per plate basis using the following equation:
$$\% rescue = 100 \times \frac{Test Well - Median Low Control}{Median High Control - Median Low Control} .$$

4. The method of claim 1, further comprising the step of determining cell count, nuclear intensity, morphology and condensation.

5. The method of claim 1, wherein the localization changes from the cytosol or mitochondria to a peroxisome.

6. The method of claim 1, wherein a candidate drug is selected from at least one of 26-Deoxymonensin B, nigericin, salinomycin, or active derivatives thereof.

7. A method of determining the effectiveness of one or more candidate pharmacoperones to treat and/or prevent protein misfolding, the method comprising:
(a) incubating the one or more candidate pharmacoperones with a first subset of the cells, and a placebo with a second subset of the cells;
(b) fixing and staining the first and second subset of cells, wherein the stain detects anti-AGT in the cells;
(c) generating images of the first and second subset of cells with a camera;
(d) measuring the co-localization of AGT with the peroxisomes in the first and second subset of cells expressing a mutant form of a peroxisomal enzyme;
(e) measuring peroxisome colocalization in the images of the first and second subset of cells; and
(f) determining if the candidate pharmacoperones modifies the colocalization of the mutant form of a peroxisomal enzyme, wherein if the candidate drug modifies the colocalization of the AGT to the peroxisome it is effective when compared to the placebo.

8. The method of claim 7, wherein the cells are AGT-mi and AGT-170 variants of a CHO-GO (glycolate oxidase) cell line.

9. The method of claim 7, wherein a range of localization values are assigned a value ranging from −1 to 1, which is the degree of overlap of the two targets with each other independent of the intensity differences of the two targets, and is calculated using the following equation:
$${r}_{p} = \frac{\sum ( x - \dot{x} ) ( y - \dot{y} )}{\sqrt{\sum {( x - \dot{x} )}^{2} {( y - \dot{y} )}^{2}}}$$
Where rₚ is the Pearson's correlation coefficient, x and y are pixel intensities of each pixel detected in the AGT and peroxisome channels, respectively, and x and y are average pixel intensities of the puncta identified as AGT and peroxisomes, respectively.

10. The method of claim 8, wherein the values are normalized on a per plate basis using the following equation:
$$\% rescue = 100 \times \frac{Test Well - Median Low Control}{Median High Control - Median Low Control}$$
wherein High Control represents the well containing AGT-mi cells treated with dimethylsulfoxide (DMSO) and Low Control represents the well containing AGT-170 cells also treated with DMSO.

11. The method of claim 7, further comprising the step of determining cell count, nuclear intensity, morphology and condensation.

12. The method of claim 7, wherein the colocalization changes from the cytosol or mitochondria to the peroxisome.

13. The method of claim 7, wherein the mutant form of the peroxisomal enzyme of pathophysiologically relevant.

14. The method of claim 7, wherein the peroxisome in the first and second subset of cells is stained with a dye, an antibody, gold labeled antibodies, ferritin labeled antibodies, peroxidase labeled antibodies, detecting perixosomal RNA, cerium, or 3,3′-diaminobenzidine.

15. The method of claim 7, wherein the mutant form of a peroxisomal is a mutant alanine: glyoxylate aminotransferase (AGT) enzyme.

16. The method of claim 7, wherein the well is part of a multi-well plate selected from 2, 4, 6, 8, 10, 12, 24, 48, 96, 394, or 1536 well plates.

17. The method of claim 7, wherein a candidate drug is selected from at least one of 26-Deoxymonensin B, nigericin, salinomycin, or active derivatives thereof.

18. A method of determining the effectiveness of a candidate drug to treating and/or prevent protein misfolding by one or more target-specific pharmacoperones, the method comprising:
(a) incubating the candidate drug to a first subset of the cells, and a placebo to a second subset of the cells;
(b) fixing and staining the first and second subset of cells, wherein the stains detects anti-alanine:glyoxylate aminotransferase (AGT) enzyme in the cells;
(c) generating images the first and second subset of cells with a camera;
(d) measuring the co-localization of AGT with the peroxisomes in a mammalian cell based system expressing a pathophysiologically relevant mislocated mutant form of a alanine: glyoxylate aminotransferase (AGT) enzyme; and
(e) determining if the candidate drug modifies the colocalization of the AGT, wherein if the candidate drug modifies the colocalization of the AGT to the peroxisome it is effective when compared to the placebo.

19. The method of claim 18, wherein the cells are AGT-mi and AGT-170 variants of a CHO-GO (glycolate oxidase) cell line.

20. The method of claim 18, wherein a range of localization values are assigned a value ranging from −1 to 1, which is the degree of overlap of the two targets with each other independent of the intensity differences of the two targets, and is calculated using the following equation:
$${r}_{p} = \frac{\sum ( x - \dot{x} ) ( y - \dot{y} )}{\sqrt{\sum {( x - \dot{x} )}^{2} {( y - \dot{y} )}^{2}}}$$
Where rₚ is the Pearson's correlation coefficient, x and y are pixel intensities of each pixel detected in the AGT and peroxisome channels, respectively, and x and y are average pixel intensities of the puncta identified as AGT and peroxisomes, respectively.

21. The method of claim 18, wherein one or more values are obtained from the imaged cells and the values are normalized on a per plate basis using the following equation:
$$\% rescue = 100 \times \frac{Test Well - Median Low Control}{Median High Control - Median Low Control}$$
wherein High Control represents the well containing AGT-mi cells treated with dimethylsulfoxide (DMSO) and Low Control represents the well containing AGT-170 cells also treated with DMSO.

22. The method of claim 18, further comprising the step of determining cell count, nuclear intensity, morphology and condensation.

23. The method of claim 18, wherein the colocalization changes from the cytosol or mitochondria to the peroxisome.

24. The method of claim 18, wherein the peroxisome in the first and second subset of cells is stained with a dye, an antibody, gold labeled antibodies, ferritin labeled antibodies, peroxidase labeled antibodies, detecting perixosomal RNA, cerium, or 3,3′-diaminobenzidine.

25. A high throughput screen for an active agent for the treatment of comprising:
plating cells comprising at least one mislocated mutant form of a peroxisomal enzyme;
adding a control and compound to each plate from a library of compounds;
fixing the cells;
contacting the cells with an agent that detects the mislocated mutant form of a peroxisomal enzyme; and
imaging the cells in the wells.

26. The screen of claim 25, wherein the cells are AGT-mi and AGT-170 variants of a CHO-GO (glycolate oxidase) cell line.

27. The screen of claim 25, wherein the dyes are selected to image the cells in the wells at 386, 485 and 549 nm to differentiate between localization of the mislocated mutant form of a peroxisomal enzyme to the mitochondria, peroxisome or cytosol.

28. The screen of claim 25, wherein the mislocated mutant form of a peroxisomal enzyme is alanine: glyoxylate aminotransferase (AGT) enzyme.

29. The screen of claim 25, wherein the mislocated mutant form of a peroxisomal enzyme is pathophysiologically relevant.

30. The screen of claim 25, wherein the agent that detects the mislocated mutant form of a peroxisomal enzyme is an anti-AGT antibody.

31. The screen of claim 25, wherein a membrane of the peroxisomes is detected with an anti-PMP70 antibody.

32. A high throughput screen for an active agent for the treatment of comprising:
plating cells comprising at least one intracellular molecule target;
adding a control and the active agent from a library of compounds to separate wells comprising the plated cells;
fixing the cells;
contacting the cells with an agent that detects the intracellular target; and
imaging the cells in the wells, wherein a difference in the intracellular localization of the intracellular target in the cells treated with a control when compared to the active agent shows that the active agent is able to change the intracellular localization of the intracellular target molecule target.

33. The screen of claim 32, wherein the intracellular target is at least one of a protein, a carbohydrate, a lipid, a nucleic acid or combinations thereof.

34. The screen of claim 32, wherein the localization changes from the cytosol or mitochondria to a peroxisome.

35. An agent capable of changing the intracellular localization of a protein selected from at least one of 26-Deoxymonensin B, nigericin, salinomycin, or active derivatives thereof.
